(19) United States
(12) Patent Application Publication
(10) Pub. No.: US 2013/0251782 A1
(43) Pub. Date: Sep. 26, 2013

(54) TOPICAL APPLICATION OF INGENOL MEBUTATE WITH OCCLUSION

(71) Applicant: LEO LABORATORIES LIMITED, Dublin (IE)

(72) Inventors: Lotte Groth Ladefoged, Ballerup (DK); Per-Ola Arvidsson, Ballerup (DK); Karsten Petersson, Ballerup (DK)

(73) Assignee: Leo Laboratories Limited, Dublin (IE)

(21) Appl. No.: 13/839,975

(22) Filed: Mar. 15, 2013

Related U.S. Application Data
(60) Provisional application No. 61/614,507, filed on Mar. 22, 2012.
(60) Provisional application No. 61/615,348, filed on Mar. 25, 2012.
(60) Provisional application No. 61/615,886, filed on Mar. 26, 2012.

(51) Int. Cl. A61K 9/70; A61K 31/22

(52) U.S. Cl. CPC A61K 9/7023; A61K 31/22
USPC 424/445; 514/549

(57) ABSTRACT

The present invention relates to formulations of ingenol mebutate and methods of preparation and use thereof. More specifically, the invention relates to formulations of ingenol-3-mebutate applied to the skin with occlusion, and methods for treating or preventing diseases or conditions.

### CROSS-REFERENCE TO RELATED APPLICATIONS

This application claims the benefit of and priority to U.S. Provisional Patent Application Nos. 61/614,507, filed Mar. 22, 2012, 61/615,348, filed Mar. 25, 2012, and 61/615,886, filed Mar. 26, 2012. The entire contents of each of the aforementioned applications are incorporated herein by reference in their entirety.

### BACKGROUND OF THE INVENTION

Ingenol-3-angelate (2-methyl-2(Z)-butenoic acid (1aR,2S,5R,5aS,6S,8aS,9R,10aR)-5,5a-dihydroxy-4-(hydroxymethyl)-1,1,7,9-tetramethyl-11-oxo-1a,2,5,5a,6,9,10,10a-octahydro-1H-2,8a-methanocyclopenta[a]cyclopropa[e]cyclodecen-6-yl ester; PEP005; ingenol mebutate)) is a protein kinase C activator that is approved in the United States for the treatment of actinic keratosis. The drug candidate has been in phase II trials for non-melanoma skin cancer [Ogbourne, S. M.; Anti-cancer Drugs, (2007), 18, 357-62].

The compound ingenol-3-angelate (PEP005) [Sayed, M. D. et.al.; Experienta, (1980), 36, 1206-1207] can be isolated from various Euphorbia species, and particularly from Euphorbia peplus [Hohmann, J. et. al; Planta Med., (2000), 66, 291-294] and Euphorbia drummondii by extraction followed by chromatography as described in U.S. Pat. No. 7449492. Certain pharmaceutical formulations of the compound have been described in WO200768963.

Angelic acid and angelic acid esters, as present in ingenol-3-angelate, are prone to isomerisation of the double bond to form the tiglate ester, particularly at basic pH [Beeby, P., Tetrahedron Lett. (1977), 38, 3379-3382, Hoskins, W. M., J. Chem. Soc. Perkin Trans. 1, (1977), 538-544, Bohlmann, F. et. al., Chem. Ber. (1970), 103, 561-563]. As a consequence only carefully optimised conditions for ester formation can be applied in the synthetic preparation of ingenol-3-angelate. Furthermore, ingenol-3-acylates are known to be unstable as they rearrange to afford the ingenol-5-acylates and ingenol-20-acylates [Sorg, B. et. al, Z Naturforsch., (1982), 37B, 748-756].

### SUMMARY OF THE INVENTION

The invention relates generally to a formulation or drug delivery composition of the compound ingenol mebutate (ingenol-3-angelate) for topical application to the skin of a subject with occlusion, and to methods of treating or preventing certain diseases or conditions using a formulation of ingenol mebutate with occlusion (i.e., with an occlusive dressing). It has now been shown that different types of occlusion will allow the active substance to penetrate to different layers in the skin.

In one aspect, the invention provides a pharmaceutical formulation comprising ingenol-3-angelate applied topically with an occlusive dressing.

In certain embodiments, the pharmaceutical formulation comprises ingenol-3-angelate in an isopropyl alcohol containing gel. In certain embodiments, the occlusive dressing is moderately breathable to nonbreathable. In certain embodiments, the occlusive film is included as a backing film in patch formulations for dermal and transdermal drug delivery

In another aspect, the invention provides a pharmaceutical formulation wherein the occlusive dressing is added immediately after the topical application of the formulation.

In another aspect, the invention provides the use of a pharmaceutical formulation of the invention for treatment of diseases requiring increased drug flux of active substance into the skin.

In certain embodiments, the disease is actinic keratosis, basal cell carcinoma, squamous cell carcinoma, photodamaged skin, serborrheic keratosis, warts or genital warts.

In certain embodiments, the occlusive dressing is added at least 15 minutes after the topical application of the formulation.

In another aspect, the invention provides the use of a pharmaceutical formulation of the invention for treatment of diseases requiring increased drug flux into the skin and penetration of active substance to a higher level in the epidermis.

In another aspect, the invention provides a method of controlling delivery of ingenol mebutate penetration to specific parts of the skin by applying variable permeable occlusion to an alcohol based pharmaceutical formulation.

In another aspect, the invention provides a method of treating or preventing a skin condition or skin disease in a subject, the method comprising:
administering a pharmaceutical formulation comprising ingenol-3-angelate
and a pharmaceutically acceptable carrier to an area of skin of the subject, and
applying an occlusive dressing to at least a portion of the area of skin of the subject.

In certain embodiments, the occlusive dressing is applied to at least 10%, 20%, 30%, 40%, 50%, 60%, 70%, 80%, 90% or 100% of the area of skin to which the pharmaceutical formulation has been applied.

In certain embodiments, the occlusive dressing comprises glass plug, Finn Chambers, aluminium foil, flexifix, flexigrid, Tegaderm, Compeed, PCDC film or parafilm.

In certain embodiments, the occlusive dressing is of variable permeability.

In certain embodiments, the occlusive dressing is applied at least 15 minutes after the topical application of the formulation.

In an embodiment the invention provides a method of treating BCC by applying ingenol mebutate followed by a non-breathable dressing as a one-time application (that is, a single application not followed by subsequent application(s) of ingenol mebutate).

In an embodiment the invention provides a method of treating BCC by applying ingenol mebutate followed by a non-breathable dressing two times.

In one aspect, the invention provides a method of treating BCC by applying PEP005 gel and a non-breathable occlusive dressing once.

In another aspect, the invention provides a topical drug delivery composition comprising ingenol-3-angelate and a pharmaceutically acceptable carrier in combination with an occlusive dressing for topical administration to the skin of a subject in need thereof.

In certain embodiments, the composition is applied to the skin of a subject with superficial basal cell carcinoma. In certain embodiments, the composition is applied to the skin of a subject with basal cell carcinoma. In certain embodiments, the composition is applied to the skin of a subject with squamous cell carcinoma. In certain embodiments, the composition is applied to the skin of a subject with actinic keratosis. In certain embodiments, the composition is applied to the skin of a subject with seborrheic keratosis. In certain embodiments, the composition is applied to the skin of a subject with genital warts. In certain embodiments, the topical drug delivery composition comprises ingenol-3-angelate in an isopropyl alcohol-containing gel. In certain embodiments, occlusive dressing is impermeable. In certain embodiments, the occlusive dressing is partially breathable. In certain embodiments, the occlusive dressing includes a backing film in a patch.

In another aspect, the invention provides a method of treating a skin disease comprising the step of administering a therapeutically effective amount of the topical drug delivery composition of the invention to the skin of a mammal. In certain embodiments, the occlusive dressing is applied immediately after topical application of the topical drug delivery composition to the area of skin. In certain embodiments, wherein the occlusive dressing is applied at least 15 minutes after topical application of the topical drug delivery composition to the area of skin. In certain embodiments, the topical drug delivery composition is applied to treat a disease requiring increased transdermal drug flux of ingenol-3-angelate. In certain embodiments, the skin disease is selected from the group consisting of actinic keratosis, basal cell carcinoma, squamous cell carcinoma, photodamaged skin, serborrheic keratosis, warts and genital warts. In certain embodiments, the epidermal drug flux of ingenol-3-angelate is increased with the occlusive dressing relative to the drug flux of ingenol-3-angelate without an occlusive dressing.

In another aspect, the invention provides a kit comprising a topical drug delivery composition of the invention.

In another aspect, the invention provides a method of treating superficial basal cell carcinoma comprising a single application of a therapeutically effective amount of the topical drug delivery composition of the invention to the skin of a mammal in need thereof. In certain embodiments, the topical drug delivery composition comprises ingenol-3-angelate in an isopropyl alcohol-containing gel. In certain embodiments, the occlusive dressing is impermeable. In certain embodiments, the occlusive dressing comprises an aluminum disk. In certain embodiments, the occlusive dressing comprises aluminum foil. In certain embodiments, wherein the occlusive dressing is partially breathable. In certain embodiments, the occlusive dressing includes a backing film in a patch. In certain embodiments, wherein the mammal is a human.

### BRIEF DESCRIPTION OF THE DRAWINGS

FIG. 1 is a graph showing the in vitro skin permeation profile of ingenol mebutate (PEP005).

FIG. 2 is a chart showing distribution of PEP005 in intact pig ear skin 21 hours after application of all test formulations, % of applied dose.

FIG. 3 is a chart showing quantities of PEP005 in applied skin (viable epidermis +dermis) at 21 hours after application, % of applied dose.

FIG. 4 is a chart showing quantities of PEP005 in receptor fluid at 21 hours after application, % of applied dose.

FIG. 5 is a graph showing in vitro skin permeation profile of PEP005.

FIG. 6 is a chart showing distribution of PEP005 in intact pig ear skin 21 hours after application, % of applied dose.

FIG. 7 is chart showing quantities of PEP005 in applied skin (viable epidermis +dermis) at 21 hours after application, % of applied dose.

FIG. 8 is a chart showing quantities of PEP005 in receptor fluid at 21 hours after application, % of applied dose.

FIG. 9 is a graph showing in vitro skin permeation profile of PEP005.

FIG. 10 is a chart showing distribution of PEP005 in intact pig ear skin 21 hours after application of all test formulations, % of applied dose.

FIG. 11 is a chart showing quantities of PEP005 in applied skin (viable epidermis+dermis) at 21 hours after application, % of applied dose.

FIG. 12 is a chart showing quantities of PEP005 in receptor fluid at 21 hours after application, % of applied dose.

FIG. 13 is a chart showing quantities of PEP005 found in the skin and in the receptor fluid 21 hours after topical application, % of applied dose.

### DETAILED DESCRIPTION OF THE INVENTION

Ingenol-3-angelate has been described as useful for treating a number of diseases. For example the compound has been described as effective in treatment of cancer, actinic keratosis, seborrheic keratosis, viral infections, bacterial infections, wound healing, and treatment of photodamaged skin.

In an embodiment of the invention the pharmaceutical formulations of the invention are contemplated for use in the treatment of superficial basal cell carcinoma (BCC), nodular BCC, squamous cell carcinoma or squamous cell carcinoma in situ (SCCIS).

In an embodiment of the invention the pharmaceutical formulations of the invention are contemplated for use in the treatment of actinic keratosis.

In an embodiment of the invention the pharmaceutical formulations of the invention are contemplated for use in the treatment of Seborrheic keratosis.

In an embodiment of the invention the pharmaceutical formulations of the invention are contemplated for use in the treatment of photodamaged skin.

In an embodiment of the invention the pharmaceutical formulations of the invention are contemplated for use in the treatment of or lesions caused by HPV infection.

In an embodiment of the invention the lesions are common warts or genital warts.

The term “cancer” in the context of the present invention is intended to cover skin cancer such as non-melanoma skin cancer, malignant melanoma, Merkel cell carcinoma, squamous cell carcinoma, basal cell carcinoma. The term “basal cell carcinoma” includes superficial basal cell carcinoma as well as nodular basal cell carcinoma. Other cancer types includes haematological cancer such as myeloid cancers in particular such as acute myeloid leukemia and chronic myeloid leukemia;

Cancer of the prostate and bladder including benign prostatic hyperplasia, prostatis intraepithelial carcinoma, carcinoma of the bladder, adenocarcinoma of the prostate and renal cell carcinoma. Other cancer include AIDS related cancer, acoustic neoma, adenocystic carcinoma, adrenocortical cancer, agnogenic myeloid metaplasia, alopecia, alveolar soft-part sarcoma, anal cancer, angiosarcoma, aplastic anaemia, astrocytoma, ataxia-telangiectasia, basal cell carcinoma (bcc), bladder cancer, bone cancers, bowel cancer, brain stem glioma, brain and CNS cancers, breast cancer, CNS cancers, carcinoid cancers, cervical cancer, childhood brain cancers, childhood cancer, childhood soft tissue sarcoma, chondrosarcoma, choriocarcinoma, colorectal cancers, cutaneous T-Cell lymphoma, dermatofibrosarcoma-protuberans, desmoplastic small round cell cancer, ductal carcinoma, endocrine cancers, endometrial cancer, ependymoma, esophageal cancer, Ewing's sarcoma, extra hepatic bile duct cancer, eye cancer, eye: melanoma, retinoblastoma, fallopian tube cancer, fanconi anaemia, fibrosarcoma, gall bladder cancer, gastric cancer, gastrointestinal cancers, gastrointestinal carcinoid cancer, genitourinary cancers, germ cell cancers, gestational trophoblastic disease, glioma, gynecological cancers, hematological malignancies, head and neck cancer, hepatocellular cancer, hereditary breast cancer, histiocytosis, Hodgkin's disease, human papillomavirus, hydatidiform mole, hypercalcemia, hypopharynx cancer, intra-ocular melanoma, isle T-cell cancer, Kaposi's sarcoma, kidney cancer, Langerhan's cell histiocytosis, laryngeal cancer, leiomyosarcoma, li-fraumeni syndrome, lip cancer, liposarcoma, liver cancer, lung cancer, lymphedema, lymphoma, Hodgkin's lymphoma, non-Hodgkin's lymphoma, male breast cancer, malignant rhabdoid cancer of kidney, medulloblastoma, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, nijmegen breakage syndrome, non-small cell lung cancer (nscic), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral neuroectodermal cancers, pituitary cancer, polycythemia vera, prostate cancer, rare cancers and associated disorders, retinoblastoma, rhabdomyosarcoma, rothmund Thomson syndrome, salivary gland cancer, sarcoma, schwannoma, sezary syndrome, small cell lung cancer (scic), small intestine cancer, soft tissue sarcoma, spinal cord cancers, stomach cancer, synovial sarcoma, testicular cancer, thymus cancer, thyroid cancer, transitional cell cancer (bladder), transitional cell cancer (renal-pelvis-/-ureter), trophoblastic cancer, urethral cancer, urinary system cancer, uroplakins, uterine sarcoma, uterus cancer, vaginal Cancer, vulva cancer, Waldenstrom's macroglobulinemia and Wilms' Cancer. The solid cancer which is treated using the methods of the present invention may be a primary lesion or may be the result of metastasis of a primary cancer. Furthermore, if the solid cancer is a metastasis of a primary cancer, the primary cancer may be either a primary solid cancer as described above or may be a dispersed primary cancer.

In an embodiment of the invention, “cancer” is skin cancer. In embodiments of the invention, skin cancer is non-melanoma skin cancer, malignant melanoma, Merkel cell carcinoma, squamous cell carcinoma, basal cell carcinoma such as superficial basal cell carcinomas or nodular basal cell carcinoma.

The term “actinic keratosis”, sometimes referred to as “solar keratosis”, in the context of the present invention is a skin condition that appears as a dry, scaly sometimes hyperkeratotic lesion, often as a result of prolonged and repeated sun or UV light exposure.

The term “photodamaged skin” in the context of the present invention is intended to cover fine lines, wrinkles and UV-aging. UV aging is often manifested by an increase in the epidermal thickness or epidermal atrophy and most notably by solar elastosis, the accumulation of elastin containing material just below the dermal-epidermal junction. Collagen and elastic fibres become fragmented and disorganised. At a cosmetic level this can be observed as a reddening and/or thickening of the skin resulting in a leathery appearance, skin fragility and irregular pigmentation, loss of tone and elasticity, as well as wrinkling, dryness, sunspots and deep furrow formation.

The term “viral infections” in the context of the present invention is intended to cover HPV infections leading to formation of warts on the body, such as the skin, genitals and mouth. HPV refers to human papilloma virus. Other viruses are selected from adeno-, papova-, herpes- (such as simplex) varicella-zoster, Epstein-Barr-, CMV-, Pox- (such as small pox-) vaccinia-, hepatitis A-, hepatitis B-, hepatitis C-, Rhino-, polio-,rubella-, arbo-, rabies-, influenza- A and B, measles-, mumps-viruses, and HIV, HTLV I and II. In an embodiment of the invention HPV infection refers to common warts or genital warts.

The term “bacterial infections” in the context of the present invention is intended to cover prokaryotic and eukaryotic bacterial infections and Gram positive and Gram negative and Gram variable bacteria and intracellular bacteria. Examples of bacteria include Treponema, Borrelia, Neisseria, Legionella, Bordetella, Escherichia, Salmonella, Shigella, Klebsiella, Yersinia, Vibrio, Hemophilus, Rickettsia, Chlamydia, Mycoplasma, Staphylococcus, Streptococcus, Bacillus, Clostridium, Corynebacterium, Proprionibacterium, Mycobacterium, Ureaplasma and Listeria. In particular the species:Treponema pallidum, Borrelia Burgdorferi, Neisseria gonorrhoea, Legionella pneumophila, Bordetella pertussis, Escherichia coli, Salmonella typhi, salmonella typhimurium, Shigella dysenteriae, Klebsiella pneumoniae, Yersinia pestis, Vibrio cholerae, Hemophilus influenza, Rickettsia rickettsii, Chlamydia trachomatis, Mycoplasma pneumonia, Staphylococcus aureus, Streptococcus pneumoniae, Streptococcus pyogenes, Bacillus anthracis, Clostridium botulinum, Clostridium tetani, clostridium perfringens, Corynebacterium diphteriae, Proprionibacterium acne, Mycobacterium tuberculosis, Mycobacterium leprae and Listeriare monocytogenes. Lower eukaryotic organism includes yeast and fungus such as Pneumocystis nerinii, Candida albicans, Aspergillus, Histoplasma capsulatum, Blastomyces dermatitidis, Cryptococcus neoformans, Trichophyton and Microsporum. Complex eukaryotic organism includes worms, insects, aracnids, nematodes, aemobe, Entamoeba histolytica, Giardia lamblia, Trichonomonas vaginalis, Trypanosoma brucei gembiense, Trypanosoma cruzi, Blantidium coli, Toxoplasma gondii, Cryptosporidium or Leishmania.

In the context of the present invention the term “wound healing” means: reducing or minimizing scar tissue or improving cosmesis or functional outcome in a wound and scar reduction, wherein the wound is cutaneous, chronic or for example diabetes associated, and includes cuts and lacerations, surgical incisions, punctures, graces, scratches, compression wounds, abrasions, friction wounds, chronic wounds, ulcers, thermal effect wounds, chemical wounds, wounds resulting from pathogenic infections, skin graft/transplant donor and recipient sites, immune response conditions, oral wounds, stomach or intestinal wounds, damaged cartilage or bone, amputation sides and corneal lesions.

The amount and/or concentration of compound in the pharmaceutical formulation is determined on the basis of the disease to be treated. For topical administration, ingenol-3-angelate may typically be present in an amount of from 0.001 to 20% by weight of the composition, such as 0.001% to about 1%. In embodiments of the present invention, the active compound (ingenol-3-angelate) is present in an amount of 0.05-1% by weight of the composition. In an embodiment of the present invention the active compound is present in 0.01-0.5% by weight of the composition. In an embodiment of the present invention the active compound is present in a concentration of around 0.1% by weight of the composition.

Penetration of the skin is facilitated by addition of penetration enhancers which include isopropyl alcohol, sulphoxides, azones, pyrrolidines, alkanols, and glycols. In embodiments of the invention, penetration enhancers include DMSO, laurocapram, 2-pyrrolidone, decanol and propylene glycol. In an embodiment of the invention, the penetration enhancer is isopropyl alcohol.

In embodiments of the invention, the therapeutically active compound (ingenol-3-angelate) is dissolved in a suitable solvent. Suitable solvents are glycols, ketone, acetates and ethers. Ingenol compounds (i.e., ingenol-3-angelate) (i.e., ingenol-3-angelate) have been shown to have good stability in alcohols such as benzyl alcohol and isopropyl alcohol. In general, ingenol compounds (i.e., ingenol-3-angelate) (i.e., ingenol-3-angelate) have previously shown to have good stability at low pH. In embodiments of the present invention, the pH of the pharmaceutical formulation is below 7. In embodiments of the present invention, the pH of the pharmaceutical formulation is below 6. In embodiments of the present invention, the pH of the pharmaceutical formulation is below 4.5. In embodiments of the present invention, the pH of the pharmaceutical formulation is below 4.0. In embodiments of the present invention, the pH of the pharmaceutical formulation is below 4.5 and no less than 2.5. In embodiments of the present invention, the pH of the pharmaceutical formulation is below 4.0 and no less than 2.5. The preferred pH range can be obtained by including an appropriate buffer. In an embodiment of the invention, the buffer is an acetate buffer, phosphate buffer or mixtures of acetate/phosphate or citrate/phosphate buffer. In embodiments of the invention, a citrate buffer is used. In embodiments of the invention a mixed citrate-phosphate buffer is used.

The ingenol compounds (i.e., ingenol-3-angelate) may be applied topically in any suitable form including solutions, emulsions (oil-in-water, water-in-oil, aerosols or foams), ointments, pastes, lotions, powders, paints, gels, hydrogels, hydrocolloids and creams, and they may be prepared so as to contain liposomes, micelles, and/or microspheres. In an embodiment the ingenol compound is applied in a gel as described in WO2007/068963 (Peplin Research PTY). In an embodiment of the invention, the gel applied can be occluded in a water proof film dressing or patch. Alternatively, the ingenol compounds (i.e., ingenol-3-angelate) may be presented in the form of an active occlusive dressing, e.g., where the ingenol compound is impregnated or coated on a dressing such as bandages, gauzes, tapes, nets, face masks, adhesive plaster, films, membranes or patches.

The term “occlusive dressing”, as used herein, refers to a dressing that, when applied to a skin surface, at least partially inhibits or prevents air or undesirable fluids from reaching a portion of the skin surface, e.g., a lesion or wound in or on the skin. An occlusive dressing can also at least partially retain medication applied to the skin. An occlusive dressing may be selected from different available types ranging from semi-occlusive (which allow some air or fluids to reach the skin surface) to fully occlusive dressings. A fully occlusive dressing can be selected, for example, from Glass plug, Finn Chambers and aluminium foil. Other occlusive dressings are types such as flexifix, flexigrid, Tegaderm and Compeed, which are all designed as breathable wound dressings with adhesive materials designed not to compromise skin surface. They also provide moderate occlusion. Alternatively, PCDC film provides moderate to high occlusion. Parafilm provides high occlusion.

The use of occlusive dressings has now been shown to improve penetration of the active substance ingenol-3-angelate.

In certain embodiments, the occlusive dressing is allowed to remain in place on the skin of the subject for a sufficient period to enhance penetration of the ingenol compound into or through the skin, e.g., for 15 minutes, 30 minutes, one hour, two hours, three hours, four hours, five hours, six hours, eight hours, ten hours, twelve hours, or 24 hours.

In an embodiment of the invention, the lesion is pretreated by an alcohol sweep before application of the pharmaceutical. The alcohol is selected from pharmaceutically acceptable alcohols such as ethanol, propanol, isopropanol etc.

The formulation of compositions and dressings contemplated herein is well known to those skilled in the art, see, for example, Remington's Pharmaceutical Sciences, 18^{th} adition, Mack Publishing, 1990.

In an embodiment of the invention, the ingenol compound may be topically applied in the form of an isopropyl alcohol-based gel. One suitable formulation includes isopropyl alcohol, benzyl alcohol, a cellulose polymer, such as hydroxyethyl cellulose, and buffer (e.g. citrate) at a pH<3. In another embodiment of the invention, the ingenol compound can be formulated for topical application in the form of a macrocetyl ether cream for example containing cetomacrogel emulsifying wax, white soft paraffin and liquid paraffin. Embodiments of the invention are disclosed and described in WO 2007/068963.

### EXAMPLES

The in vitro skin permeation and penetration of PEP005 was investigated with various types of occlusion dressings, using flow through diffusion cells. Dressings were applied immediately (without formulation drying), as well as after the formulation had dried. Additionally, PEP005 might also be absorbed into the occlusion dressing, which also was investigated.

Methods: The in vitro permeation and penetration of PEP005 into pig ear skin was studied using PermeGear® flow through diffusion cells (n=6 per formulation). The skin diffusion experiment was allowed to proceed for 21 hours into a recipient phase consisting of 0.04 M isotonic phosphate buffer pH 7.4. Samples from the different skin layers and recipient phase were collected in order to determine flux and skin distribution of PEP005. The samples were analysed by LC-MS/MS.

The test formulation used is an aqueous based gel formulation containing; PEP005 0.5 mg/g, Benzyl Alcohol 9 mg/g, Isopropanol 300 mg/g, Citric acid 5.4 mg/g, Sodium Citrate dihydrate 1.4 mg/g, Hydroxyethyl Cellulose 15 mg/g and water up to 1 g, with a formulation pH of 3.2.

The following occlusion dressings were investigated:

Study PMPN1017
Glass plug

Study PMPN1024
Finn chamber
Flexifix (flexigrid), Opsite
Tegaderm

Study PMPN1028
Flexifix (flexigrid), Opsite
Aluminium foil
PVDC (poly vinyl di-chloride), Coloplast
Compeed,
Parafilm (M model)

Finn chamber and Aluminium foil are potential dressings that may be considered inert against PEP005 absorption. Finn Chambers are previously used “LEO in-house” in irritation studies. Aluminium foil was chosen due to the flexibility around a treated lesion as in comparison to the stiff aluminium cup of the Finn chamber. Aluminium foil and Finn chamber are considered to give full occlusion. Flexifix/flexigrid, Tegaderm and Compeed are known breathable wound dressings with adhesive material (glue) that are designed to not compromise skin surface at the point of removal. Additionally, they are considered to give moderate occlusion. PCDC film from Coloplast is used as backingfilm in patch formulations and is considered to give moderate to high occlusion.

Parafilm is documented in the literature as being used as occlusive dressing. The Glass plug could be considered a diffusion cell control, representing full occlusion and most likely inert against PEP005 absorption. Aluminium foil, Parafilm, PVDC and Compeed was fixed to the treatment area with Flexifix.

### 1.1 Skin Membrane

Full thickness skin from pig ears was used in the study. The ears were kept frozen at −18° C. until use. Before the experiment, the ears were placed in a refrigerator (2-8° C.) for slow defrosting. The hairs were removed using a veterinary hair trimmer. The skin was cleaned for subdermal fat using a scalpel, and two pieces of skin were cut from each ear. The pieces of skin were refrozen and kept for no longer than 4 weeks. On the day of the experiment, the ears were placed in a refrigerator (2-8° C.) for slow defrosting. The skin was mounted on diffusion cells in a balanced order. Applied PEP005 gel formulation was let to dry (Isopropanol evaporation) for about 10 minutes prior to occlusion dressing application. Dressings were placed over the skin and mounted between the donor and receptor chamber.

### 1.2 Diffusion Cell

The used PermeGear® automated system incorporate nine flow through diffusion cells (1-9) made of clear glass, in which the donor and receptor chambers were separated by a diffusion membrane and held together by a pinch clamp. The flow through cells had an available diffusion area of 3.14 cm² and receptor volumes ranging between 11.1 to 12.2 ml. The specific volume was measured and registered for each cell. The membrane was placed over a support with an orifice of 2 cm in diameter. The inlet and outlet ports of the receptor chamber were connected to stainless steel HPLC tubing and the cells were placed in a cell warmer connected with a Haake®-DC10 circulating bath programmed to 38° C., resulting in a temperature on the membrane surface of 32° C. To ensure adequate stirring, the cells were placed on a magnetic stirrer (default=500 rpm). Furthermore the cells were connected to a 12-channel peristaltic pump, Ismatec® IPC-12 and the receptor fluid was pumped continuously through each cell to be collected in centrifuge vials of glass with a round bottom placed at an Isco® Retriver IV fraction collector. An Indexing Controller was used to program independently the duration of each shuttle in the retriever. After mounting the skin, physiological saline (35° C.) was filled into the receptor chamber for hydration of the skin. After half an hour the saline was replaced by receptor medium (35° C.) and left for hydration another hour.

### 1.3 Receptor Fluid

0.04 M isotonic phosphate buffer pH 7.4 containing 4% bovine serum albumin (BSA) was used as receptor fluid. The receptor fluid was degassed in an ultrasound water bath for 10 minutes prior to the start of the experiment. It was ensured that sink conditions were present at all times during the study period, i.e. that the concentration of the drug compounds in the recipient phase was below 10% of the solubility of the drug compound in the medium.

### 1.4 Application, Dosage and Volume of Test Formulation

Prior to application of the formulation, the experiment was started by starting the pump. The formulation was applied to the skin membrane at 0 hours and in a finite dose of 4 mg/cm² corresponding to 15-20 mg formulation per cell. The formulation was applied using a glass spatula and the residual amount of formulation was determined, thus giving the actual amount of formulation applied to the skin.

### 1.5 Sampling Times and Lag Time

The pump was set at a flow rate of 1.9 ml/h. About 6 ml of the receptor fluid was sampled in centrifuge vials of glass with a round bottom from each cell every third hour until 21 hours post application. Due to the continuous sample collecting, the cells were automatically refilled with new receptor medium. This influenced the sampling times, which were therefore different for each individual cell. Furthermore, the sample collection of the first 45 minutes was discarded due to the lag time of the system.

The skin diffusion experiments were allowed to proceed for 21 hours. At 21 hours the excess formulation was removed on the skin surface using cotton swab and two times tape stripping (Transpore® tape). The stratum corneum was removed by use of D-squame® tape discs (Curaderm,US). The applied skin containing the viable epidermis and dermis was isolated and analyzed. The Receptor fluid was collected and analysed

### 1.6 Study Design

The in vitro skin permeation and penetration of PEP005 from the test formulations were tested in 6 replicates, i.e. n=6. The study was balanced.

### 1.7 Analysis of Test Substances and Data Processing

The concentration of PEP005 in the samples was determined by means of LC-MS/MS. Standard curves in the concentration interval of 0.03-300 ng/ml were prepared in mobile phase (for non-absorbed formulation and skin surface samples), recipient fluid (recipient fluid samples) and in skin homogenate (applied and non-applied skin samples), respectively. An internal standard of EO1271 was used.

For the penetration data: The distribution of compound in non-absorbed formulation and skin surface, in applied and non-applied skin and in the receptor fluid after 21 hours was calculated and expressed as ng/cm² and % of applied dose. For the permeation data: Based on the obtained concentrations and the amount of recipient phase withdrawn at each specific time, the cumulative permeated amount of drug substance was calculated for each diffusion cell and plotted as a function of time. For all individual cells, the flux was determined from the slope of the linear part of the curve using linear regression analysis.

### 2 Results and Discussion

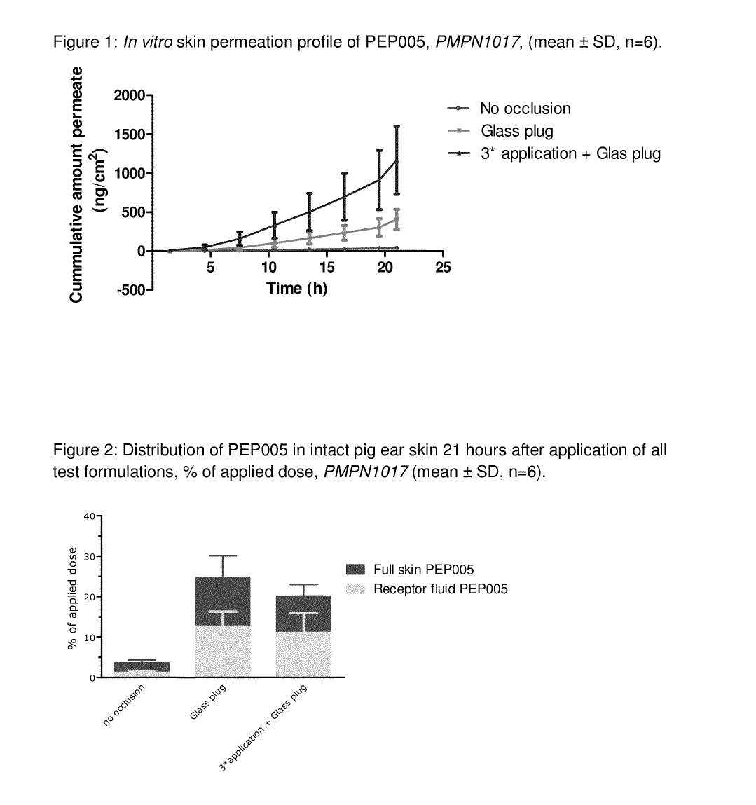

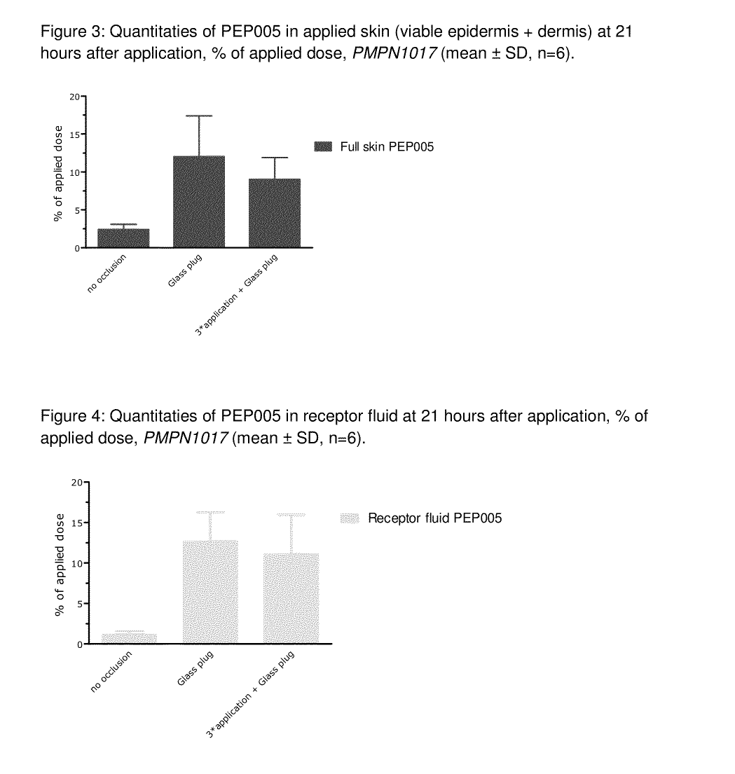

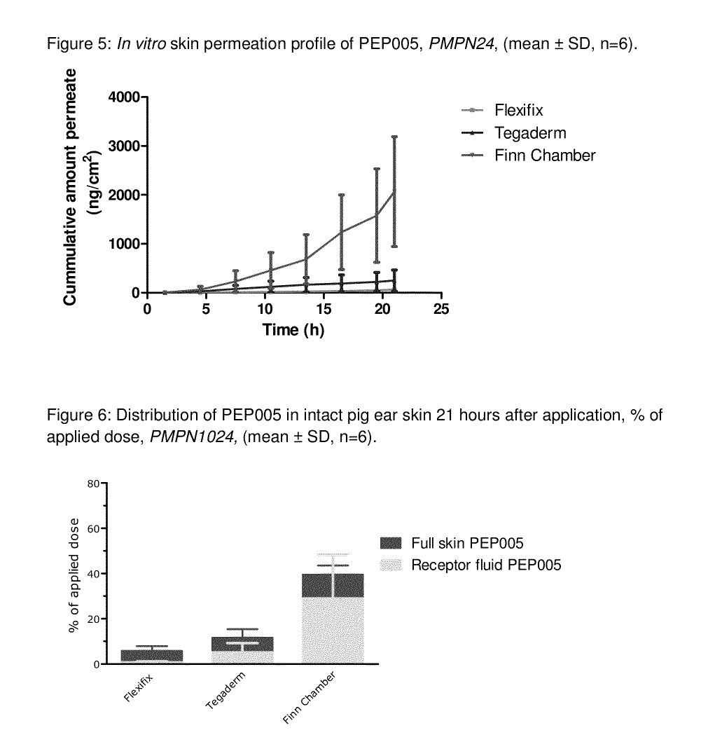

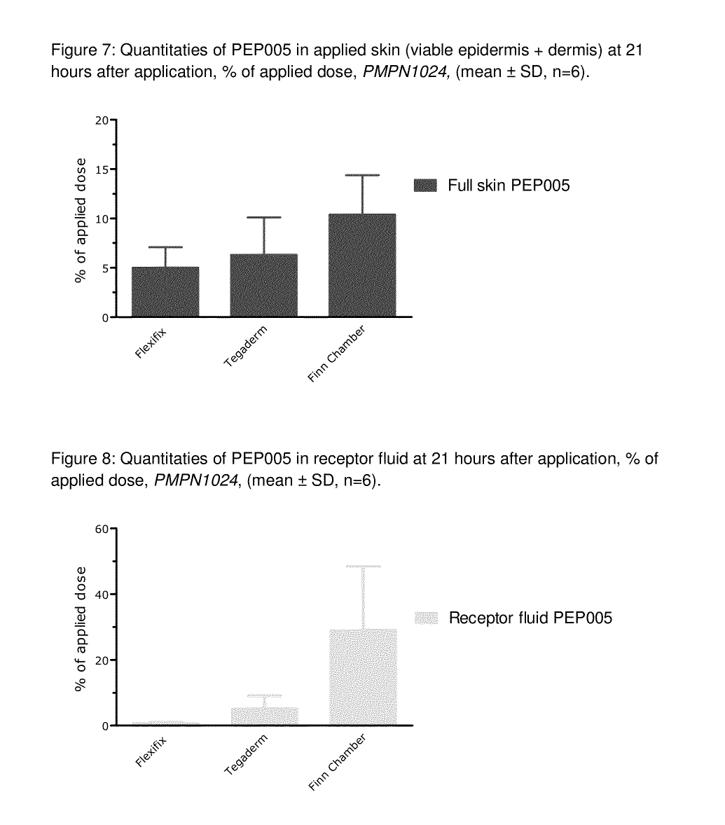

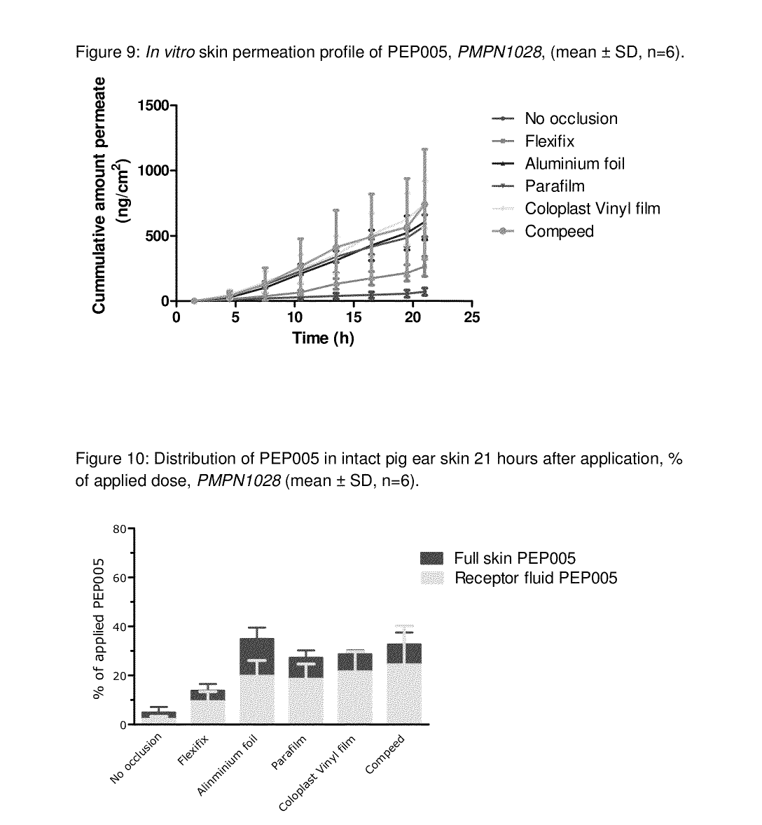

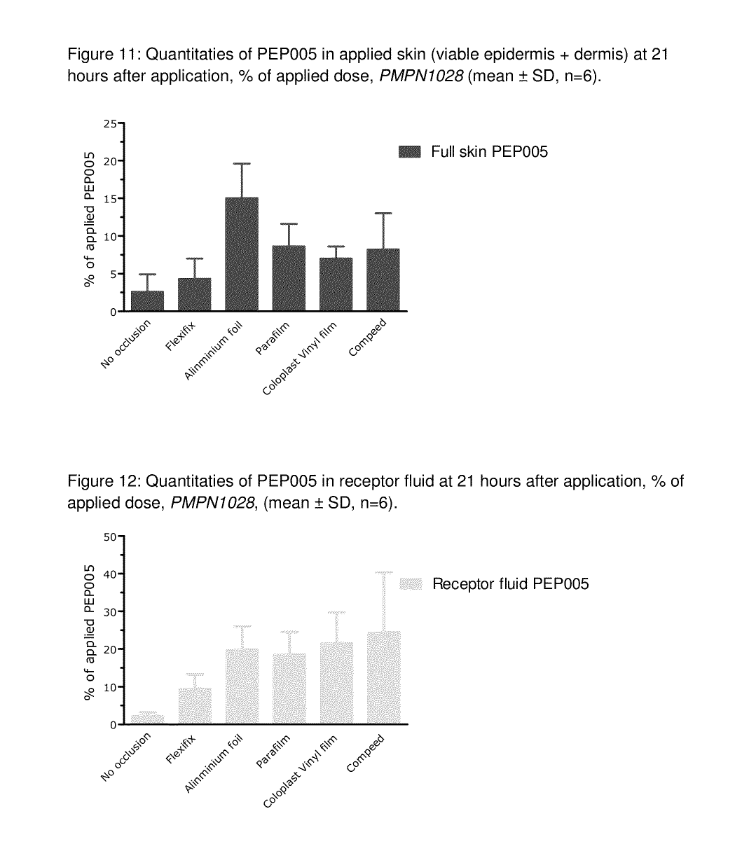

The in vitro skin permeation profiles of PEP005 from the investigated formulations are shown in FIGS. 1, 5 and 9. The total permeated amounts and the steady state fluxes are presented in Table 2. The relative distribution or quantities of PEP005 at 21 hours post application is illustrated as % of applied dose in FIG. 2-4, 6-9, 10-12. Table 3 lists the quantities of PEP005 found in the applied skin and in the receptor fluid at 21 h after topical application on intact skin. Table 4 list the extracted amount of PEP005 from dressings used in study PMPN1028.

Study PMPN1017

The amount of PEP005 increased significantly both in the skin and in the receptor medium after occlusion using the glass plug, in comparison to the non-occluded treatment (Tables 2 and 3). Additional increase in amount of PEP005 was observed after three successive applications of PEP005. The formulation was left to dry between each application of the three applications. The flux of PEP005 through the skin was increased by the occlusion of the glass plug and the two additional applications of PEP005 gel formulation. The flux was highest after three times application under occlusion.

Study PMPN1024

The occlusive effect of Tegaderm increased the skin penetration and permeation in comparison to the occlusion by Flexifix, though only the effect on the skin permeation was significant. Both dressings are considered to induce moderate occlusive effect. The amount of PEP005 in the skin when using the Finn Chamber was higher compared to Flexifix and Tegaderm (Table 3). Furthermore, the amount of PEP-005 in the receptor fluid was considerably larger than Flexifix and Tegaderm. The full occlusion of the Finn Chamber resulted in the highest delivery of PEP005 to the skin.

Study

By using the semi-occlusive dressing Flexifix, a 3 fold increase in skin permeation was observed compared to the non-occluded treatment (Table 2). The amount of PEP005 in the skin and in the receptor fluid using Flexifix tended to be higher compared to the amount found in study PMPN1024. The aluminium foil, parafilm, Coloplast vinyl film and Compeed induced 9-10 fold increase in permeation of PEP005 compared to the non-occluded treatment. The permeation was comparable with the permeation of PEP005 after occlusion with Finn Chamber investigated in an earlier study.

The penetrated amount of PEP005 was increased 2-6 fold after occlusion compared to the non-occluded treatment. The aluminium foil tended to induce higher penetration of PEP005 compared to the other types of dressing. The fully occlusive dressings in this study resulted in highest delivery of PEP005 to the skin. However, the moderately occlusive Compeed induced similar skin delivery of PEP005 as the fully occlusive dressings.

The amount of PEP005 absorbed in the dressings after the end of the experiment was highest for Flexifix (Table 4). However, the extraction method for PEP005 from the dressings was not validated.

In conclusion, the skin delivery of PEP005 to the skin was increased using an occlusive dressing over the PEP005 gel test area. Higher skin penetration and permeation of PEP005 were obtained by fully occlusive dressings.

### 3 Tables

TABLE 1
Test formulation

| | Concentration | |
|---|---|---|
| Formulation | mg/g | Batch no. |
| PEP005 gel | 0.5 | CBA-C |

TABLE 2
In vitro skin permeation results for PEP005 (mean ± SD, n = 6).

| | | | |
|---|---|---|---|
| | | Total permeated | Flux at steady |
| | | amount at 21 | state |
| Formulation | Dressing | hours (ng/cm²) | (ng/cm²/hour) |

Study PMPN1017

| | | | |
|---|---|---|---|
| PEP005 gel | None | 37.0 ± 13.0 | 2.5 ± 0.8 |
| PEP005 gel | Glass plug | 387 ± 126 | 29.9 ± 7.1 |
| PEP005 gel | Glass plug | 1015 ± 456 | 77.0 ± 25.5 |
| 3 times application | | | |

Study PMPN1024

| | | | |
|---|---|---|---|
| PEP005 gel | Flexifix | 225 ± 99 | 5.0 ± 2.1 |
| PEP005 gel | Tegaderm | 38.0 ± 13.0 | 11 ± 8.8 |
| PEP005 gel | Finn Chamber | 1721 ± 1072 | 172 ± 86 |

Study PMPN1028

| | | | |
|---|---|---|---|
| PEP005 gel | None | 68 ± 27 | 4.0 ± 1.7 |
| PEP005 gel | Flexifix | 252 ± 75 | 16.9 ± 5.0 |
| PEP005 gel | Aluminium foil | 582 ± 136 | 37.9 ± 5.9 |
| PEP005 gel | Parafilm | 553 ± 83 | 29.7 ± 6.2 |
| PEP005 gel | Coloplast Vinyl | 705 ± 182 | 49.7 ± 11 |
| | film | | |
| PEP005 gel | Compeed | 701 ± 407 | 39.6 ± 22.3 |

TABLE 3
Quantities of PEP005 found in the skin and in the
receptor fluid 21 hours after topical application,
ng/cm² (% of applied dose), mean ± SD, n = 6.

| | | | |
|---|---|---|---|
| | | Total skin | Receptor |
| | | ng/cm² (% of | fluidng/cm² |
| Formulation | Dressing | applied) | (% of applied) |

Study PMPN1017

| | | | |
|---|---|---|---|
| PEP005 gel | none | 75 ± 19 | 37 ± 13 |
| | | (2.4 ± 0.7) | (1.2 ± 0.4) |
| PEP005 gel | Glass plug | 352 ± 149 | 387 ± 126 |
| | | (11.9 ± 5.4) | (12.7 ± 3.6) |
| PEP005 gel | Glass plug | 845 ± 288 | 1015 ± 456 |
| 3 times application | | (9.2 ± 2.9) | (11.1 ± 4.9) |

Study PMPN1024

| | | | |
|---|---|---|---|
| PEP005 gel | Flexflix | 225 ± 99 | 38 ± 13 |
| | | (5.0 ± 2.1) | (0.8 ± 0.3) |
| PEP005 gel | Tegaderm | 278 ± 165 | 240 ± 180 |
| | | (6.3 ± 3.8) | (5.3 ± 3.9) |
| PEP005 gel | Finn Chamber | 631 ± 236 | 1721 ± 1027 |
| | | (10.4 ± 4.0) | (29.1 ± 19.5) |

Study PMPN1028

| | | | |
|---|---|---|---|
| PEP005 gel | None | 71 ± 54 | 68 ± 27 |
| | | (2.6 ± 2.3) | (2.3 ± 1.0) |
| PEP005 gel | Flexifix | 114 ± 60 | 252 ± 75 |
| | | (4.3 ± 2.) | (9.5 ± 3.9) |
| PEP005 gel | Aluminium foil | 441 ± 98 | 582 ± 136 |
| | | (15 ± 4.6) | (19.9 ± 6.2) |
| PEP005 gel | Parafilm | 258 ± 53 | 553 ± 83 |
| | | (8.6 ± 3.0) | (18.6 ± 6.0) |
| PEP005 gel | Coloplast Vinyl | 237 ± 55 | 705 ± 182 |
| | film | (1.4 ± 0.9) | (21.6 ± 8.2) |
| PEP005 gel | Compeed | 235 ± 107 | 701 ± 407 |
| | | (8.2 ± 4.8) | (24.5 ± 15.9) |

TABLE 4
Extraction of PEP005 from dressing after experiment

| | | Extraction of |
|---|---|---|
| | | PEP005 from |
| | | dressings % of |
| Formulation | Dressing | applied dose |
| PEP005 gel | None | |
| PEP005 gel | Flexifix | 5.1 ± 3.3 |
| PEP005 gel | Aluminium foil | 0.5 ± 0.5 |
| PEP005 gel | Parafilm | 0.5 ± 0.4 |
| PEP005 gel | Coloplast Vinyl | 1.4 ± 0.9 |
| | film | |
| PEP005 gel | Compeed | 1.9 ± 1.3 |

TABLE 5
Quantities of PEP005 found in the skin and in the
receptor fluid 21 hours after topical application,
ng/cm² (% of applied dose), mean ± SD, n = 6.

| | Dressing and | Total skin | Receptor fluid |
|---|---|---|---|
| | application of | ng/cm² (% of | ng/cm² (% of |
| Formulation | dressing | applied) | applied) |
| PEP005 gel | None | 62 ± 18 | 44 ± 20 |
| | | (0.8 ± 0.5) | (0.5 ± 0.2) |
| PEP005 gel | Double | 84 ± 31 | 151 ± 92 |
| | Tegaderm | | |
| | After drying | (1.0 ± 0.4) | (1 7 ± 1.1) |
| PEP005 gel | Double Flexifix | 74 ± 66 | 117 ± 114 |
| | After drying | (0.8 ± 0.7) | (1.3 ± 1.2) |
| PEP005 gel | Double Flexifix | 104 ± 23 | 257 ± 198 |
| | Without drying | (1.2 ± 0.4) | (3.0 ± 2.6) |
| PEP005 gel | Finn chamber r | 59 ± 43 | 141 ± 155 |
| | After drying | (1.0 ± 0.7) | (2.3 ± 2.6) |
| PEP005 gel | Finn chamber | 86 ± 26 | 447 ± 230 |
| | Without drying | (1.6 ± 0.3) | (8.5 ± 4.3) |

### Results

The skin delivery of PEP005 was increased using occlusive dressing over the PEP005 gel test area compared to non-occlusion. Skin penetration and permeation also were increased by occlusion, including semi-occlusive and fully occlusive dressings. Highest skin penetration and permeation of PEP005 were obtained by fully occlusive dressings. Enhanced skin delivery was obtained when the occlusive dressing was applied immediately after gel formulation application. In particular, results obtained showed that penetration to the deeper parts of the skin were enhanced when full occlusion was applied immediately after application of the PEP005 gel.

Fully occluded samples showed greater penetration overall compared to the non-occluded and additionally showed greater penetration to the dermis compared to the epidermis.

The amount of PEP005 increased significantly both in the skin and in the receptor medium after occlusion using the glass plug in comparison to the non-occluded treatment. Additional increase in amount of PEP005 was observed after three successive applications of PEP005 in conjunction with using the glass plug.

The occlusive effect of Tegaderm increased the skin penetration and permeation in comparison to the occlusion by Flexifix, though only the effect on the skin permeation was significant. The amount of PEP005 in the skin when using Finn Chamber was higher compared to Flexifix and Tegaderm.

By using the semi-occlusive dressing Flexifix, a 3-fold increase in skin permeation was observed when compared to the non-occlusive treatment. The aluminium foil, parafilm, Coloplast vinyl film and Compeed induced 9-10 fold increase permeation of PEP005 compared to the non-occlusive treatment.

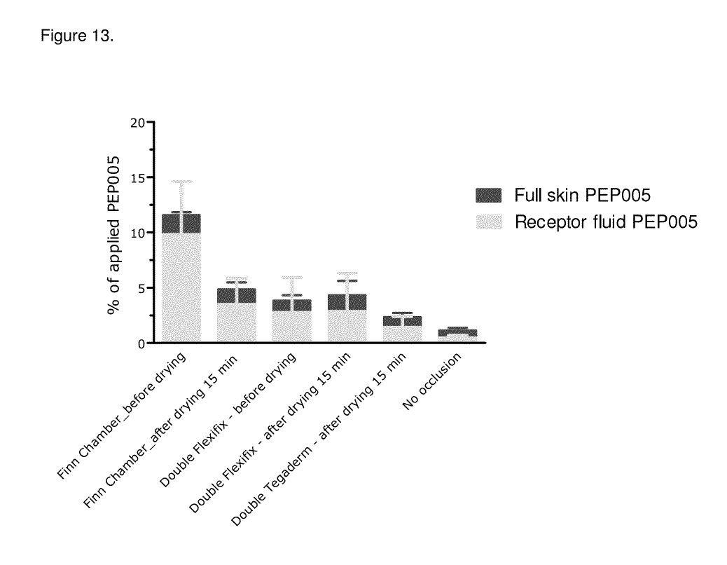

As seen in Table 5 and FIG. 13, higher skin permeation of PEP005 was found without drying of the gel formulation compared to having the formulation dry for 15 minutes before occlusion was initiated, both for Flexifix and Finn chamber. The difference in skin permeation between without drying and drying was higher for the Finn chamber (full occlusion) compared to Flexifix (semi-occlusion). Applying occlusion immediately after applying the formulation will not allow the isopropyl alcohol to evaporate but rather enhances permeation by promoting penetration of PEP005 through the skin.

The results of all of the experiments and data obtained and discussed above indicate that, due to increased permeation and penetration of ingenol angelate after topical application to an area of skin followed by application of an occlusive dressing to the area of skin, a lower dose of ingenol angelate may be applied when the area of skin is occluded after application than would be used without occlusion. Additionally, fewer applications may be needed for obtaining the same results as obtained without an occlusive dressing. Diseases which have lesions in the deeper parts of the skin, such as skin cancers sBCC and SCC, may in particular benefit from the present invention. Also, skin with stronger barrier properties such as hyperkeratotic actinic keratosis is likely to benefit from application of an occlusive dressing to the treated area of skin.

The above hypothesis was tested in a clinical trial:

PEP005 Gel, 0.05% has been well tolerated when used on trunk and extremities for NMSC (non-melanoma skin cancer) in previous clinical trials and no safety concerns have been identified. Efficacy, measured as histological clearance, has been evaluated in 2 previous trials involving sBCC and it did not seem possible to increase the efficacy by increasing the concentration. Therefore, in an effort to maintain the safe and well tolerated profile of the PEP005 Gel, 0.05% application, as well as increase efficacy, the treatment regimen for the trial included prolongation of the treatment regimen to 3 consecutive days with or without occlusion of the treatment area.

Hence, the purpose of this trial was to investigate if treatment, once daily for up to 3 consecutive days, with PEP005 Gel, 0.05%, with or without occlusion is safe and tolerated in patients with sBCC lesions on the trunk and extremities.

The primary objective of the clinical trial was to evaluate the safety of PEP005 Gel, 0.05% when administered for up to 3 consecutive days application (Day 1, 2 and 3), to a sBCC lesion on the trunk and extremities.

Another objective of the clinical trial was to evaluate the efficacy of PEP005 Gel, 0.05% when administered for up to 3 consecutive days application (Day 1, 2 and 3), to a sBCC lesion on the trunk and extremities.

This was a phase 2, multi-centre, parallel group, open-label trial to evaluate the safety and efficacy of PEP005 (ingenol mebutate) Gel, 0.05%, when administered for up to 3 consecutive days (Day 1, 2 and 3) to a sBCC lesion on the trunk or extremities.

The trial population was planned to include 75 subjects aged at least 18 years with a primary diagnosed and histologically confirmed sBCC located on the trunk or extremities which was suitable for excision.

In the context of the present invention and clinical trial eligible subjects were:

### 9.3.1 Inclusion Criteria

1. Patient is male or female and at least 18 years of age

2. A primary diagnosed and histologically confirmed sBCC located on the trunk or extremities which is suitable for excision

the biopsy should be conducted no more than 60 days prior to the screening visit

the biopsy specimen should have removed no more than 20% of the total tumour mass

3. The longest axes of the sBCC lesion between 4-15 mm

4. Female patients must be of either:

Non-childbearing potential, provided there is a laboratory confirmed serum follicle stimulating hormone level 40m IU/ml or there is a confirmed clinical history of sterility (e.g., the patient is without a uterus); or

Childbearing potential, provided there are negative urine pregnancy test results prior to study treatment, to rule out pregnancy

5. Women of childbearing potential (WOCBP) must be willing to consent to using effective contraception at trial entry and for the duration of trial participation. Effective contraception is defined as follows:

Injectable or implantable hormones;

Intrauterine device;

Trans-abdominal surgical sterilisation;

Sterilisation implant device;

Surgical sterilisation of male partner;

Complete abstinence from sexual intercourse for 2 weeks before exposure to trial medication and throughout the clinical trial

6. Patient has the ability to understand the nature of the trial and agree to comply with the prescribed dosage regimens, report for regularly scheduled trial visits and communicate to trial personnel about adverse events and concomitant medication use

7. Patient has provided informed consent documented by signing the Informed Consent Form prior to any trial-related procedures, including any alteration of medications in preparation for trial entry

8. Written consent has been obtained for examination and storage of tissue from biopsy and excised sBCC lesion

9. Patient has agreed to allow photographs of the selected treatment area to be taken and used as part of the trial data package

10. Agreement to keep the selected treatment area dry and occluded (untouched/undisturbed) for 24 hours following PEP005 Gel application, if applicable

Exclusion Criteria for the present clinical trial:

1. Location of the sBCC lesion:

within 10 cm of an incompletely healed wound (including any residual scabbing from the lesion biopsy);

on the hand or foot;

on the breast of women;

on the anogenital area;

within 10 cm of a suspected BCC, SCCIS or SCC; and/or

within 5 cm of an area/lesion that has been previously treated with PEP005 (ingenol mebutate) Gel

2. History of recurrence of the sBCC lesion

3. Histological evidence of nBCC, SCCIS or SCC in the biopsy specimen.

4. Selected sBCC lesion thicker than 1 mm

5. Histological evidence of sBCC with micro-nodular, cystic, keratotic, infundibulocystic, metatypical, or sclerosing (desmoplastic or morphoeic) features; sBCC with perineural involvement in the biopsy specimen; or underlying dermatofibroma

6. History or evidence of skin conditions other than the trial indication that would interfere with evaluation of the trial drug (e.g., eczema, unstable psoriasis, xeroderma pigmentosa)

7. Clinically diagnosed or suspected disease that suppresses the immune system (e.g., human immunodeficiency virus or hepatitis)

8. Clinically diagnosed or suspected uncontrolled systemic disease (e.g., hypertension, diabetes)

9. Any abnormal vital signs at Baseline that are medically significant or would impact the safety of the patient or the interpretation of the trial results, as determined by Investigator clinical judgment

10. Clinical diagnosis/history or evidence of any medical condition that would expose a patient to an undue risk of a significant AE or interfere with assessments of safety and efficacy during the course of the trial, as determined by Investigator clinical judgment

11. Anticipated need for in-patient hospitalisation or in-patient surgery. Note that cosmetic/therapeutic procedures are not excluded if they fall outside of the criteria detailed in Table 2: Prohibited Treatments and Procedures during the Trial

12. Diagnosis of xeroderma pigmentosa or Gorlin Syndrome (i.e. Basal Cell Naevus Syndrome)

13. Known sensitivity or allergy to any of the ingredients in PEP005 (ingenol mebutate) Gel.

14. Anticipated excessive or prolonged exposure to ultraviolet light (e.g. sunlight, tanning beds)

15. Enrolment or participation in a clinical research trial within 30 days of entry into this trial

Prohibited Therapies and/or Medications: within 2 weeks prior to the Screening visit

16. Cosmetic or therapeutic procedures (e.g., use of liquid nitrogen, surgical excision, curettage, dermabrasion, medium or greater depth chemical peel, laser resurfacing): within 2 cm of the selected treatment area

17. Use of acid-containing therapeutic products (e.g., salicylic acid or fruit acids, such as alpha and beta hydroxy acids and glycolic acids), topical retinoids or light chemical peels: within 2 cm of the selected treatment area

18. Use of topical moisturisers/creams/lotions (non-medicated/non-irritant salves are acceptable), artificial tanners or topical steroids: within 2 cm of the selected treatment area

Prohibited Therapies and/or Medications: within 4 weeks prior to the Screening visit

19. Treatment with immuno-modulators (e.g., azathioprine), cytotoxic drugs (e.g., cyclo-phosphamide, vinblastine, chlorambucil, methotrexate, podophyllin, camptothecin) or interferon/interferon inducers

20. Treatment with systemic medications that suppress the immune system (e.g., cyclosporine, prednisone, methotrexate, alefacept, infliximab)

21. Treatment/therapy with UVB

Prohibited Therapies and/or Medications: within 8 weeks prior to the Screening visit

22. Treatment with 5-FU, imiquimod, diclofenac, or photodynamic therapy: within 2 cm of the selected treatment area

Prohibited Medications: within 6 months prior to the Screening visit

23. Use of systemic retinoids (e.g., isotretinoin, acitretin, bexarotene)

Eligible subjects were randomised to receive physician-applied trial medication in one of the following 3 treatment groups:

Group 1) PEP005 Gel, 0.05%, up to 3 consecutive days treatment, occluded with an aluminium disk;

Group 2) PEP005 Gel, 0.05%, up to 3 consecutive days treatment, occluded with an Opsite™ disk;

Group 3) PEP005 Gel, 0.05%, up to 3 consecutive days treatment, not occluded.

The trial consisted of 4 periods (screening, treatment, follow-up and post-study follow-up) which are briefly described below.

### Screening Period (Day -28 to -2)

The screening visit was to occur between Day -28 and -2. At the screening visit the Informed consent procedure was conducted as described in the protocol, Appendix 16.1.1, and subjects were screened for eligibility.

### Treatment Period (Days 1, 2 and 3)

Eligible subjects received PEP005 Gel on Days 1, 2 and 3. Each subject was assessed prior to each treatment on Days 2 and 3 as to whether they were tolerating the trial medication (e.g., did not have LSRs or AEs that would preclude treatment in the investigators opinion). If tolerability to trial medication was maintained, the subjects received all 3 doses of PEP005 Gel, 0.05%, regardless of whether the sBCC lesion being treated was resolved or not.

Follow-Up Period (Days 2, 3 (as applicable), 4 (as applicable), 8, 15, 29, 57, 85 and 120)

Subsequent follow-up visits for safety assessments were made on Days 2, 3 (as applicable), 4 (as applicable), 8, 15, 29, 57, 85 and 120. Histological and clinical efficacy assessments were only conducted at Day 120. If trial medication was not administered on Day 2 a follow-up visit on Day 3 or 4 was not necessary, unless clinically indicated. If trial medication was not administered on Day 3 a follow-up visit on Day 4 was not necessary, unless clinically indicated.

### Post-Study Follow-Up Period

Post-excision wound evaluation visit was conducted 5 to 14 days after the excision visit. Post-study follow-up visits were required every 7 to 28 days for all subjects with unresolved treatment-related AEs at Day 120. Subjects were followed until either resolution or assessed as being clinically stable.

Subjects received one of the following treatments:

1) PEP005 Gel, 0.05%, up to 3 consecutive days treatment, occluded with an aluminium disk

2) PEP005 Gel, 0.05%, up to 3 consecutive days treatment, occluded with an OpSite™ disk

3) PEP005 Gel, 0.05% up to 3 consecutive days, not occluded

Subjects were randomly assigned to receive either occlusive material or no occlusive material.

The volume of PEP005 Gel applied to the selected treatment area was 30 μl for lesions with the longest axes of between 4-10 mm and 50 μl for lesions with the longest axes of between 11-15 mm.

The actual treatment analysis set included the same subjects as in the full analysis set, i.e. 75 randomised subjects. However, 2 subjects who received the wrong treatment were assigned to their actual treatment group, i.e. PEP005 Aluminium Disk.

Thus, in the actual treatment analysis set 27 subjects were included in PEP005 Aluminium Disk group and 24 subjects both in PEP005 Opsite™ Disk group and PEP005 No Occlusion group.

| | PEP005 | | PEP005 | | | |
|---|---|---|---|---|---|---|
| | Aluminium | | OpSite ™ | | PEP005 No | |
| | Disk | | Disk | | Occlusion | |
| | (n = 27) | | (n = 24) | | (n = 24) | |
| Complete | Number | | Number | | Number | |
| clinical | of | | of | | of | |
| clearance | subjects | % | subjects | % | subjects | % |
| Complete | 20 | 74.1 | 18 | 75.0 | 18 | 75.0 |
| clearance | | | | | | |
| Non-complete | 7 | 25.9 | 6 | 25.0 | 6 | 25.0 |
| clearance | | | | | | |
| Total | 27 | 100.0 | 24 | 100.0 | 24 | 100.0 |
| Lower 95% CL | | 53.7 | | 53.3 | | 53.3 |
| (complete | | | | | | |
| clearance) | | | | | | |
| Upper 95% CL | | 88.9 | | 90.2 | | 90.2 |
| (complete | | | | | | |
| clearance) | | | | | | |

Overall, 60% of subjects (45 of 75) received treatment once daily for 3 consecutive days. The number of doses received per treatment group is shown below. Within the

Aluminium Disk group, only 3 subjects (11.1%) received all 3 doses of trial medication, 2 subjects (7.4%) received 2 doses and 22 subjects (81.5%) were only treated once. Within the Opsite™ Disk group, 19 subjects (79.2%) received all 3 doses of trial medication and the remaining 5 subjects (20.8%) were treated for 2 days. In the No Occlusion group, 23 subjects (95.8%) received all 3 doses of trial medication, with 1 subject (4.2%) receiving 2 doses.

| | PEP005 | | PEP005 | | | |
|---|---|---|---|---|---|---|
| | Aluminium | | OpSite ™ | | PEP005 No | |
| | Disk | | Disk | | Occlusion | |
| | (n = 27) | | (n = 24) | | (n = 24) | |
| | Number | | Number | | Number | |
| Number of doses | of | | of | | of | |
| received | subjects | % | subjects | % | subjects | % |
| 1 | 22 | 81.5 | 0 | 0.0 | 0 | 0.0 |
| 2 | 2 | 7.4 | 5 | 20.8 | 1 | 4.2 |
| 3 | 13 | 11.1 | 19 | 79.2 | 23 | 95.8 |
| Total | 27 | 100.0 | 24 | 100.0 | 24 | 100.0 |

The aim of this phase 2 trial was to evaluate safety and efficacy of PEP005 Gel, 0.05% when administered for up to 3 consecutive days to sBCC lesion on the trunk and extremities, occluded either with an aluminium disk or an Opsite™ disk or not occluded.

The clinical clearance rate was similar across all 3 treatment groups.

Histological analysis is regarded as the gold standard for the evaluation of the treatment effect in sBCC. In daily practice the efficacy evaluation is, however, usually made clinically, wherefore the clinical clearance and the composite endpoint of histologic as well as clinical clearance are important endpoints. In this regard, in the Aluminium Disk group the complete histological clearance rate was 70.4%, the clinical clearance rate was 74.1% and the composite clearance 63.0%, indicating a high agreement between histologic and clinical assessment of clearance. The observed efficacy in the patients in the Aluminium Disk group who received only one dose of PEP005 gel in combination with occlusion was unexpected given that the purpose of the study was to test whether efficacy could be increased with once daily dosing for 3 consecutive days using occlusion.

Across all treatment groups, LSRs were most pronounced in the days immediately following application. LSRs were more elevated in the Aluminium Disk group and peaked at Day 3 compared to Day 4 in the other groups. Mean composite values returned to baseline at Day 57 in the Aluminium Disk group and Day 29 in the other 2 groups. At Day 120 all 3 groups were similar and below baseline.

The contents of all patents, patent applications, and publications mentioned in this specification are herein incorporated by reference to the same extent as if each independent patent and publication was specifically and individually indicated to be incorporated by reference.

What is claimed is:

1. A pharmaceutical formulation comprising ingenol-3-angelate applied topically with an occlusive dressing to an area of skin of a subject.

2. The pharmaceutical formulation of claim 1 which comprises ingenol-3-angelate in an isopropyl alcohol containing gel.

3. The pharmaceutical formulation of claim 1-2, wherein the occlusive dressing is moderately breathable to nonbreathable.

4. The pharmaceutical formulation of claim 3, wherein the occlusive dressing includes a backing film in a patch for dermal and transdermal drug delivery.

5. The pharmaceutical formulation of claims 1-3, wherein the occlusive dressing is applied immediately after topical application of the pharmaceutical formulation to the area of skin.

6. The pharmaceutical formulation of claims 1-3, wherein the occlusive dressing is applied at least 15 minutes after topical application of the pharmaceutical formulation to the area of skin.

7. The use of a pharmaceutical formulation according to claim 1, wherein the pharmaceutical formulation is applied to treat a disease requiring increased transdermal drug flux of ingenol-3-angelate.

8. The use of a pharmaceutical formulation according to claim 7, wherein the disease is selected from the group consisting of actinic keratosis, basal cell carcinoma, squamous cell carcinoma, photodamaged skin, serborrheic keratosis, warts and genital warts.

9. The use of a pharmaceutical formulation according to claim 7, wherein the epidermal drug flux of ingenol-3-angelate is increased with the occlusive dressing relative to the drug flux of ingenol-3-angelate without an occlusive dressing.

10. The use of a pharmaceutical formulation according to claim 5, wherein the pharmaceutical formulation is applied to treat a disease selected from the group consisting of actinic keratosis, basal cell carcinoma, squamous cell carcinoma, photodamaged skin, serborrheic keratosis, warts, and genital warts.

11. A method of controlling ingenol-3-angelate penetration in the skin, such method comprising the application of an occlusive dressing and an alcohol-based pharmaceutical formulation of ingenol-3-angelate to the skin.

12. The method of claim 11, wherein the occlusive dressing is of variable permeability.

13. The method of claim 11, wherein the occlusive dressing is applied at least 15 minutes after the topical application of the formulation

14. A method of treating or preventing a skin condition or skin disease in a subject, the method comprising:
administering a pharmaceutical formulation comprising ingenol-3-angelate
and a pharmaceutically acceptable carrier to an area of skin of the subject, and
applying an occlusive dressing to a least a portion of the area of skin of the subject.

15. The method of claim 14, wherein the skin disease or skin condition is selected from the group consisting of actinic keratosis, basal cell carcinoma, squamous cell carcinoma, photodamaged skin, serborrheic keratosis, warts, and genital warts.

16. The method of claim 14, wherein the occlusive dressing is selected from the group consisting of a glass plug, Finn Chamber, aluminium foil, flexifix, flexigrid, Tegaderm, Compeed, PCDC film and parafilm.

17. The method of claim 14, wherein the occlusive dressing is of variable permeability.

18. The method of claim 14, wherein the occlusive dressing is applied at least 15 minutes after the topical application of the formulation

19. A method of treating BCC by applying PEP005 gel and a non-breathable occlusive dressing once.

20. A topical drug delivery composition comprising ingenol-3-angelate and a pharmaceutically acceptable carrier in combination with an occlusive dressing for topical administration to the skin of a subject in need thereof.

21. The topical drug delivery composition of claim 20, wherein the composition is applied to the skin of a subject with superficial basal cell carcinoma.

22. The topical drug delivery composition of claim 20, wherein the composition is applied to the skin of a subject with basal cell carcinoma.

23. The topical drug delivery composition of claim 20, wherein the composition is applied to the skin of a subject with squamous cell carcinoma.

24. The topical drug delivery composition of claim 20, wherein the composition is applied to the skin of a subject with actinic keratosis.

25. The topical drug delivery composition of claim 20, wherein the composition is applied to the skin of a subject with seborrheic keratosis.

26. The topical drug delivery composition of claim 20, wherein the composition is applied to the skin of a subject with genital warts.

27. The topical drug delivery composition of claim 20, which comprises ingenol-3-angelate in an isopropyl alcohol-containing gel.

28. The topical drug delivery composition of claim 20, wherein the occlusive dressing is impermeable.

29. The topical drug delivery composition of claim 20, wherein the occlusive dressing is partially breathable.

30. The topical drug delivery composition of claim 20, wherein the occlusive dressing includes a backing film in a patch.

31. A method of treating a skin disease comprising the step of administering a therapeutically effective amount of the topical drug delivery composition of claim 20 to the skin of a mammal.

32. The method of claim 31, wherein the occlusive dressing is applied immediately after topical application of the topical drug delivery composition to the area of skin.

33. The method of claim 31, wherein the occlusive dressing is applied at least 15 minutes after topical application of the topical drug delivery composition to the area of skin.

34. The method of claim 31, wherein the topical drug delivery composition is applied to treat a disease requiring increased transdermal drug flux of ingenol-3-angelate.

35. The method of claim 31, wherein the skin disease is selected from the group consisting of actinic keratosis, basal cell carcinoma, squamous cell carcinoma, photodamaged skin, serborrheic keratosis, warts and genital warts.

36. The method of claim 31, wherein the epidermal drug flux of ingenol-3-angelate is increased with the occlusive dressing relative to the drug flux of ingenol-3-angelate without an occlusive dressing.

37. A kit comprising the topical drug delivery composition of claim 20.

38. A method of treating superficial basal cell carcinoma comprising a single application of a therapeutically effective amount of the topical drug delivery composition of claim 20 to the skin of a mammal in need thereof.

39. The method of claim 38, wherein the topical drug delivery composition comprises ingenol-3-angelate in an isopropyl alcohol-containing gel.

40. The method of claim 38, wherein the occlusive dressing is impermeable.

41. The method of claim 40, wherein the occlusive dressing comprises an aluminum disk

42. The method of claim 40, wherein the occlusive dressing comprises aluminum foil.

43. The method of claim 38, wherein the occlusive dressing is partially breathable.

44. The method of claim 38, wherein the occlusive dressing includes a backing film in a patch.

45. The method of claim 38, wherein the mammal is a human.
